(19) United States
(12) Patent Application Publication
(10) Pub. No.: US 2024/0240739 A1
(43) Pub. Date: Jul. 18, 2024

(54) HYGIENIC FITTING FOR SANITARY PROCESSING EQUIPMENT

(71) Applicant: Feldmeier Equipment, Inc., Syracuse, NY (US)

(72) Inventors: Kyle A. Brown, Jamesville, NY (US); Colby W. Clark, Manlius, NY (US); Tyler E Robillard, Oswego, NY (US)

(73) Assignee: Feldmeier Equipment, Inc., Syracuse, NY (US)

(21) Appl. No.: 18/289,296

(22) PCT Filed: Jun. 2, 2022
(86) PCT No.: PCT/US22/31942

§ 371 Date: Nov. 2, 2023

Related U.S. Application Data
(60) Provisional application No. 63/196,014, filed on Jun. 2, 2021.

(51) Int. Cl. F16L 55/115

(52) U.S. Cl. CPC F16L 55/115

(57) ABSTRACT

A hygienic cap is provided for sealing a fitting, the hygienic cap includes a rim portion defining a mating surface that is configured to receive a compliant gasket along the mating surface. A central cover portion is integrally formed with the rim portion and defines a cavity having a threshold radius defined by an arcuate internal surface of the cavity. The threshold radius inhibits stagnation of a cleaning fluid along the arcuate internal surface during cleaning operations.

### CROSS REFERENCE TO RELATED APPLICATIONS

This application claims the benefit of the filing date and the priority of U.S. Provisional Patent Application No. 63/196,014, filed on Jun. 2, 2021.

### BACKGROUND

The processing of fluid products in the food, dairy, and pharmaceutical industries often employ a system of tanks, vessels, pumps, pipe runs, and fluid conduits for the storage, transfer, and mixing of various ingredients used in production. Such processing systems are typically outfitted with various fittings for monitoring a variety of processing parameters such as temperature, pressure, flow rate, conductivity, pH, etc., of the system as the ingredients are stored, moved and/or mixed. Accordingly, such tanks, vessels, and piping runs often include a plurality of ferrules, flanges, or tee fittings which form dead legs or dead ends when caps or instruments are attached. A dead leg is defined as an area of entrapment in a vessel or pipe run where system design and operating conditions result in insufficient process fluid flow, presenting a risk for particulate, chemical, or biological contamination (ASME BPE 2019 GR-8). Similarly, a dead end may be defined as an area or space wherein a product, ingredient, cleaning or sanitizing agent, or other extraneous matter may be trapped, retained, or not completely displaced during operational or cleaning procedures (3-A Sanitary Standard for General Requirements 00-01).

Inasmuch as such food, dairy, and pharmaceutical products must be free of harmful bacteria, it will be appreciated that the processing equipment must be periodically cleaned, sanitized, and/or sterilized, i.e., flushed with a cleaning solution or steam, to remove residue and bacteria from the internal cavities, chambers, and conduits of the equipment. The ASME Bioprocessing Equipment (BPE) Standards is the industry guide for the design, manufacture, and maintenance of processing equipment used in the production of biopharmaceuticals where a defined standard of purity and bioburden control is required. The 3-A Sanitary Standards define the general requirements for sanitary (hygienic) equipment intended for processing milk, milk products, foods, food ingredients, beverages, or other edible materials. Of particular concern in each of these hygienic standards are internal areas where cleaning fluids either, cannot access, or flow with sufficiently high velocity, to properly clean the subject area.

In FIG. 1, a cross-sectional view of a typical Tee Fitting or dead-end 10 includes a cap 14, a flanged ferrule 16 projecting orthogonally from a fluid conduit 18, and a compliant gasket 20 disposed between the cap 14 and the flanged ferrule 16. More specifically, the compliant gasket 20 is disposed between a first plane 24 defined along the underside of the cap 14 and a second plane 26 defined by the upper surface of the flanged ferrule 16. Furthermore, a clamp 28 engages a tapered outer surface 14R of the cap 14, i.e., along its outer rim, and a is disposed over the cap 14 and the flange of the ferrule 16 to compress and seal the cap and ferrule 14, 16 against the compliant gasket 20.

A cleaning fluid, depicted by arrows 30, flows at high velocity through the fluid conduit 18 to evacuate the conduit 18 and dead-end 10 of residual product and bacteria remaining after a production cycle. Inasmuch as the dead-end 10 is perpendicular to the direction of fluid flow, the fluid velocity within the dead-end 10 is significantly diminished by comparison to the flow within the conduit 18. A further examination of the internal geometry reveals that the flow rate stagnates, or approaches zero, at the intersection or corner between the cap 14 and the compliant gasket 20. It will be appreciated, that the geometry of prior art dead-end fittings 10 inhibits the rate of fluid velocity which increases the difficulty to adequately clean the interior of the fittings 10.

To address the flow deficiencies, the BPE and the 3-A Sanitary Standards requires all internal angles less than one hundred and thirty-five degrees (135°) must maximize the radius in corner areas for ease of cleanability. When certain threshold radii cannot be achieved, such as when sealing flat surfaces and flow control apertures, e.g., dead-end fittings having planar or flat-end cap attachments, an exception is made. That is, for fittings having hygenic unions or joints consisting of: (i) two neutered ferrules having flat faces with a concentric groove, and (ii) a mating gasket 20 secured by a hygienic clamp having non-protruding, recess-less contact surfaces, the only suggested requirement pertains to the ease of joint disassembly to facilitate cleanability of the ferrule 16 and associated cap 14. The 3-A Standards make a similar exception for flat-end cap attachments.

In view of the foregoing, the BPE and 3-A relax the requirements for dead-end fittings having planar or flat-end cap attachments inasmuch as the geometry appears to present an unsolvable problem. Consequently, prior art dead-end fittings must be held to minimum extension, while cleaning times, temperatures and/or fluid velocity increased, in an effort to clean the problem areas where residue collects and bacteria has an opportunity to grow.

A need, therefore, exists for a new and useful sealing interface between a dead-end fitting and a sealing cap which facilitates high-velocity fluid flow to adequately clean the internal surfaces of residue and harmful bacteria.

### BRIEF SUMMARY OF THE DISCLOSURE

A hygienic cap is provided for sealing a fitting comprising a rim portion having a mating surface configured to receive a compliant gasket along the mating surface, and a central cover portion integrally formed with the rim portion. The hygienic cap, furthermore, has a cavity defining a cylindrical bore and an arcuate internal surface enclosing the cylindrical bore. The arcuate internal surface defines a threshold radius configured to prevent stagnation of a fluid induced to flow through the fitting and along the arcuate internal surface during cleaning operations.

In another embodiment, a hygienic cap is provided for sealing a fitting having a cylindrical bore defining a fitting diameter. The hygienic cap comprises a rim portion having a mating surface configured to receive a compliant gasket along the mating surface, and a central cover portion integrally formed with the rim portion. The cover portion defines a cavity having: (i) a cap diameter corresponding to the gasket diameter, (ii) a threshold length; and a (iii) a threshold radius defined by an arcuate internal surface enclosing the cavity. The threshold length of the cavity permits cleaning fluid to flow across the compliant gasket while minimizing head loss in the cleaning fluid. The threshold radius prevents stagnation, i.e., prevents the flow velocity from dropping below a threshold velocity, of the fluid flow along the arcuate internal surface during cleaning operations. A minimum flow velocity ensures that residual product will not serve as a source of bacterial growth.

In another embodiment, a hygienic cap for sealing a fitting includes a rim portion defining a mating surface configured to receive a compliant gasket along the mating surface and a central cover portion integrally formed with the rim portion. The cover portion defines a cavity having a threshold radius defined by an arcuate internal surface of the cavity. The threshold radius is configured to inhibit stagnation of a cleaning fluid along the arcuate internal surface during cleaning operations.

In an embodiment, the cavity of the cover portion further defines a threshold length structured to enable the cleaning fluid to flow across the compliant gasket while minimizing head loss in the cleaning fluid. In an embodiment, the cavity further defines a cap diameter that corresponds to a gasket diameter. In an embodiment, the threshold radius is greater than about at least one-quarter inches (¼ or 0.25 inches). In another embodiment, the threshold radius is greater than about at least one sixteenth inches ( 1/16 or 0.0625 inches). In an embodiment, the threshold length is greater than about at least one-sixteenth inches ( 1/16 or 0.0625 inches). In a further embodiment, the threshold length is greater than about at least one-thirty seconds of an inch ( 1/32 or 0.03 inches).

Another embodiment of a hygienic cap for sealing a fitting includes a rim portion defining a mating surface configured to receive a compliant gasket along the mating surface and a central cover portion integrally formed with the rim portion. The cover portion forms a cavity defining at least one obtuse angle along an internal surface of the cavity. The at least one obtuse angle is configured to inhibit stagnation of a cleaning fluid along the arcuate internal surface during cleaning operations. In an embodiment, the hygienic cap further comprises a second obtuse angle adjacent the first obtuse angle, formed by and between a first sloping surface along the underside of the central cover portion and a cylindrical bore along an internal face of the cavity. In an embodiment, the second obtuse angle is greater than the first obtuse angle. In an further embodiment, the first obtuse angle is greater than about one-hundred and thirty-five degrees (135°) and the second obtuse angle is greater than about two-hundred and twenty-five degrees (225°).

An embodiment of a method of manufacturing a hygienic cap for sealing a fitting includes the steps of: (1) structuring a rim portion to define a mating surface to receive a compliant gasket along the mating surface; structuring a central cover portion to be integrally formed with the rim portion; and structuring the central cover portion to define a cavity comprising a threshold radius defined by an arcuate internal surface of the cavity such that the threshold radius is configured to inhibit stagnation of a cleaning fluid along the arcuate internal surface during cleaning operations.

In another embodiment, a method is provided for cleaning a hygienic fitting comprising the steps of:
(a) configuring a hygienic cap to include a rim portion having a mating surface for receiving a compliant gasket, the compliant gasket having a gasket diameter corresponding to the fitting diameter;
(b) integrally forming a central cover portion in combination with the rim portion, the cover portion defining a cavity having: (I) a cap diameter corresponding to the gasket diameter, (ii) a threshold length, and (iii) a threshold radius defined by an arcuate internal surface enclosing the cavity;
(c) configuring the threshold length such that the cavity permits a cleaning fluid to flow across the compliant gasket while minimizing head loss in the cleaning fluid; and
(d) configuring the threshold radius such that stagnation of the cleaning fluid along the arcuate internal surface is prevented during cleaning operations.

The above embodiments are exemplary only. Other embodiments as described herein are within the scope of the disclosed subject matter.

### BRIEF DESCRIPTION OF THE DRAWINGS

So that the manner in which the features of the disclosure can be understood, a detailed description may be had by reference to certain embodiments, some of which are illustrated in the accompanying drawings. It is to be noted, however, that the drawings illustrate only certain embodiments and are therefore not to be considered limiting of its scope, for the scope of the disclosed subject matter encompasses other embodiments as well. The drawings are not necessarily to scale, emphasis generally being placed upon illustrating the features of certain embodiments. In the drawings, like numerals are used to indicate like parts throughout the various views, in which:

FIG. 1 depicts a sectional view of a prior art dead-end fitting showing areas of problematic fluid flow when inducing a flow of cleaning fluid into the fitting from a connecting fluid conduit;

FIG. 2 depicts a perspective view of a conventional fluid processing tank or vessel having a plurality of dead-end fittings which may be sealed with caps, sight glasses, safety sensors, or instrumentation to enable system monitoring such as temperature, pressure and pH measurement;

FIG. 3 depicts a cross sectional view through a first embodiment of a cap according to the teachings of the present disclosure, which cap is configured to mitigate areas of problematic fluid flow when inducing a flow of cleaning fluid into a dead-end fitting;

FIG. 4 depicts a cross sectional view through a second embodiment of a cap according to the teachings of the present disclosure;

FIG. 5 depicts a partially broken-away sectional view through a third embodiment of a cap according to the teachings of the present disclosure, which cap includes at least one obtuse angle along the internal face of the cavity to mitigate areas of problematic fluid flow when inducing a flow of cleaning fluid into a dead-end fitting;

FIG. 6 depicts a partially broken-away sectional view through a fourth embodiment wherein an internal cavity defines a plurality of obtuse angles along the rim and central portions of the cap;

FIG. 7 is an isolated perspective view of a ferrule nozzle for use in combination with a perspective view of a containment including a cap, a ferrule nozzle, compliant gasket and a compression clamp;

FIG. 8 is an isolated perspective view of the compliant gasket;

FIG. 9 is an isolated perspective view of the ferrule cap;

FIG. 10A is an isolated perspective view of an assembled fitting for insertion into an aperture through the tank or vessel, including the cap, the ferrule nozzle, the compliant gasket and a compression clamp;

FIG. 10B is an exploded view of the assembled fitting of FIG. 10A; and

FIG. 11 is a schematic sectional view of a cap according to the teachings of the present invention and the fluid flow in the cavity of the cap during cleaning operations.

Corresponding reference characters indicate corresponding parts throughout several views. The examples set out herein illustrate several embodiments, but should not be construed as limiting in scope in any manner.

### DETAILED DESCRIPTION

The present disclosure relates to sanitary and hygienic equipment, and more particularly, fittings associated therewith, which provide cleanliness such that products produced by such equipment will not adversely affect human or animal health. While the disclosure describes a dead-end fitting used in combination with a tank, vessel or piping run, it will be appreciated that the teaching provided herein relate to any ferrule, fitting, nozzle, tap, T-, Y-, elbow fitting, etc. used to access the contents of any plumbing, conduit, or container system or equipment.

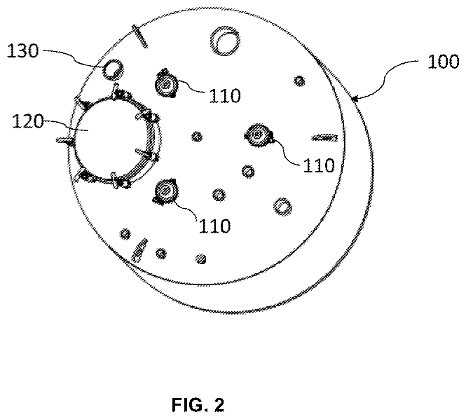

In FIG. 2, a conventional fluid processing tank or vessel 100 has a plurality of dead-end fittings 110 for effecting fluid connections which may be used to access or enable system monitoring such as through the use of temperature, pressure and/or pH sensors or gauges. The container 100 may include a variety of fittings 110 such as a large personal access port 120 and smaller nozzle fittings 130.

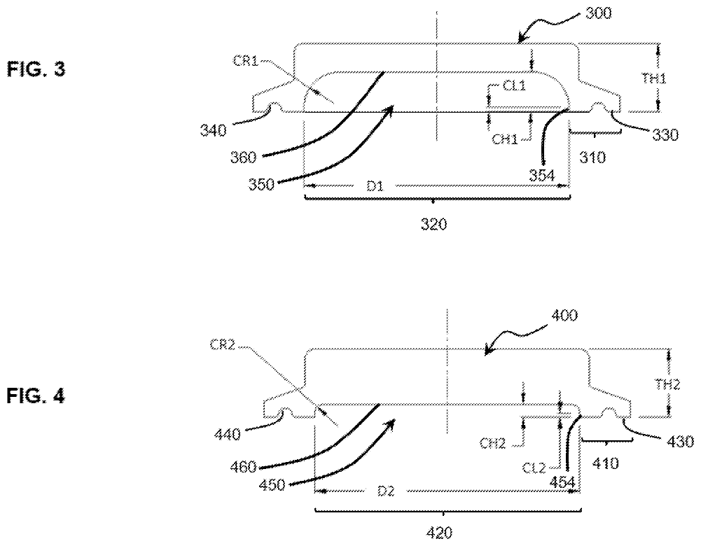

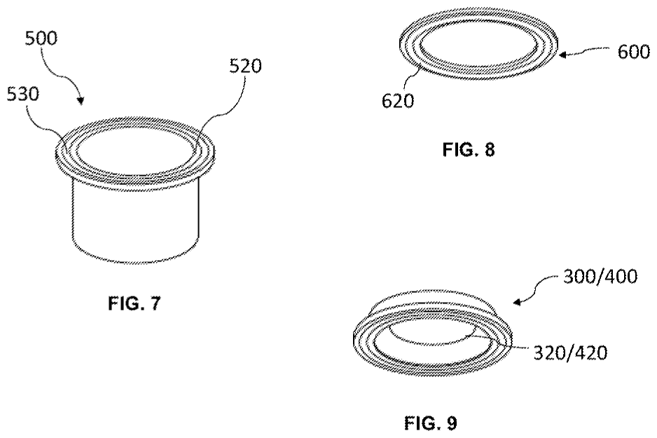

FIGS. 3-6 depict several embodiments which illustrate the teachings set forth in the present disclosure. Therein, hygienic caps 300, 400, 370, and 470 are geometrically configured to mitigate areas of problematic fluid flow when inducing a flow of cleaning fluid into a fitting 110. More specifically, each of the caps 300, 400, 370, and 470 may be configured for use in combination with a ferrule nozzle 500 shown in FIG. 7. In the broadest sense of the disclosure, each of the caps 300, 400, 370, and 470 define an internal cavity 350, 450, 375, and 475, respectively, geometrically configured to mitigate stagnation of fluid flow along the respective internal surface 360, 460, 378, and 488. In one embodiment shown in FIGS. 3 and 7-10B, the hygienic cap 300 comprises a rim portion 310 and a central portion 320 integrally formed with, and covering, the rim portion 310. Furthermore, the rim portion 310 of the hygienic cap 300 has a mating surface 330 which is configured to receive a compliant gasket 600 (see FIG. 8). The compliant gasket 600 functions to provide a non-protruding, recess-less, seal between the hygienic cap 300 and the ferrule nozzle 500 (see FIG. 7). In the described embodiment, the rim portion 310 may include a circular groove 340 for receiving a circular protrusion 620 disposed on the face of the compliant gasket 600. The circular protrusion 620 augments the efficacy of a seal produced between the compliant gasket 600 and the rim portion 310 of the hygienic cap 300 and laterally retains the compliant gasket 600 relative thereto.

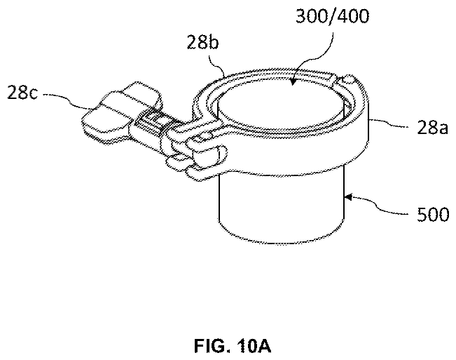

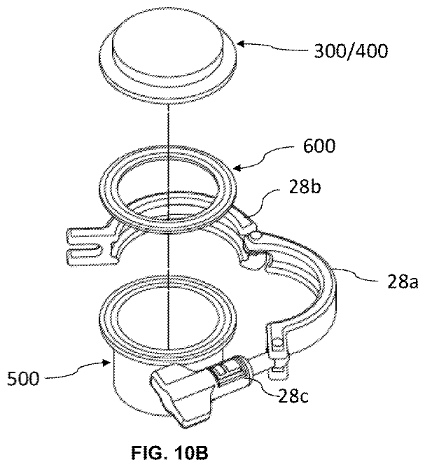

Similarly, the compliant gasket 600 may include a circular protrusion (not shown) on the underside of the gasket 600, i.e., on the side facing the flange 520 of the ferrule nozzle 500 (FIG. 7), for engaging a circular groove 530 formed or machined into the flange 520. Once again, the groove 530 functions to augment sealing and lateral retention of the compliant gasket 600. In FIGS. 10A-B, a cap 300, 400, 370, 470, configured in accordance with the teachings of the present invention, is disposed in combination with a ferrule nozzle 500. A compliant gasket (not seen in the figure) seals the cap 300, 400, 370, 470 to the ferrule nozzle 500 by means of a compression clamp 28a, 28b. That is, the compression clamp 28a, 28b applies a radial load to effect a compressive axial load on the cap 300, 400, 370, 470 to seal the compliant gasket against the ferrule nozzle 500. In an embodiment, the compression clamp 28a, 28b may further include a clamp lock 28c configured to secure the clamp in a locked state so as to continuously apply a radial load to effect an compressive axial load on the cap 300, 400, 370, 470 to seal the compliant gasket against the ferrule nozzle 500.

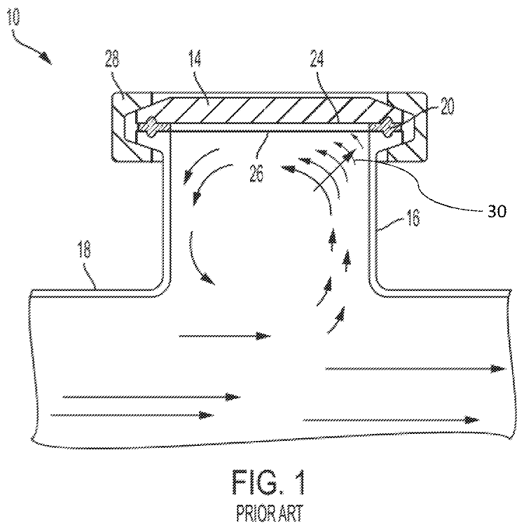

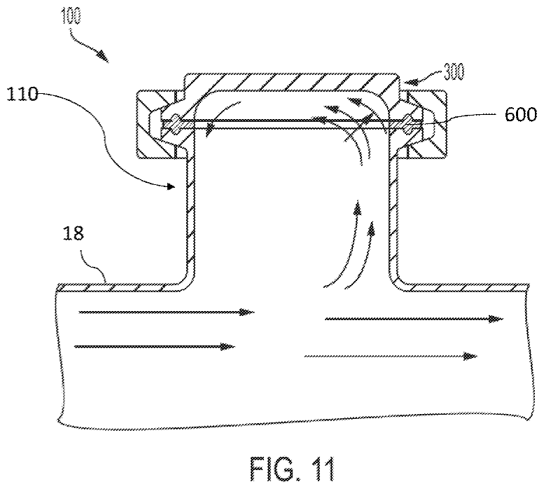

Returning to FIG. 3, the cap 300 includes a cavity 350 defined by a cylindrical bore 354 having a threshold axial length CH1 and an arcuate internal surface 360 defined by a threshold radius CR1 for enclosing the cylindrical bore 350. More specifically, in the described embodiment, the cylindrical bore 354 has a bore diameter D1 which corresponds with the bore diameter of the ferrule nozzle 500. The arcuate internal surface 360, and particularly, the threshold radius CR1 at the upper corners of the cavity 350, is configured to prevent stagnation of a product or cleaning fluid induced to flow into the cavity 350 through the ferrule nozzle 500. The threshold length CH1 of the cavity 350 permits the cleaning fluid to flow across the compliant gasket 600 while minimizing head loss in the cleaning fluid. Furthermore, the threshold radius CR1 prevents stagnation of the cleaning fluid along the arcuate internal surface 360 during cleaning operations. FIG. 11 depicts the cleaning fluid 30 (shown by arrows) flow in the cavity 350 of the cap 300 wherein it will be appreciated that the flow velocity remains essentially constant across the arcuate internal surface 360 of the cavity 350. In contrast to FIG. 1, the cleaning fluid 30 flows at high velocity through the fluid conduit 18 (or container) and dead-end 110 with little to no loss of fluid velocity within the dead-end 110. Moreover, the high fluid velocity is maintained across the compliant gasket 600 to adequately clean the internal dead-end surfaces of residue and harmful bacteria.

In the embodiment depicted in FIG. 3, the hygienic cap 300 defines a threshold radius CR1 which is greater than at least about one-quarter inches (¼″ or 0.25″) and a threshold length CL1 of about one-sixteenth inches ( 1/16″ or 0.0625″). In the embodiment shown in FIG. 4, the hygienic cap 400 defines a threshold radius CR2 which is greater than at least about one-sixteenth inches ( 1/16″ or 0.0625″) and a threshold length CL2 of about one thirty-second inches ( 1/32″ or 0.03″).

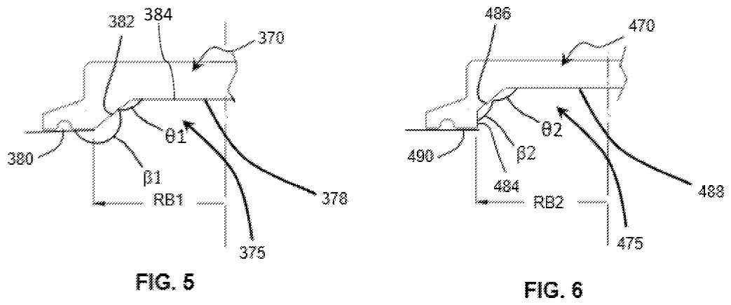

While the embodiments of FIGS. 3 and 4 each depict a cavity 350, 450 defined by a cylindrical bore 354, 454 and an arcuate internal surface 360, 460, it will be appreciated that other embodiments are contemplated wherein the internal surfaces of the cavity are configured to prevent or mitigate fluid stagnation along the respective internal surfaces. For example, FIG. 5 depicts a cap 370 having a cavity 375 defining an internal surface 378 configured to produce at least one obtuse angle θ1. In the described embodiment, a first obtuse angle θ1 is produced by and between a sloping surface 382 and an underside surface 384 of the cavity 375. A second obtuse angle β1 may be produced by and between a mating plane 380 of the rim portion of the cap and the sloping surface 382 of the internal cavity 375. In the described embodiment, the second obtuse angle β1 is greater than the first obtuse angle θ1. More specifically, the first obtuse angle θ1 is greater than about one-hundred and thirty-five degrees (135°) while the second obtuse angle β1 is greater than about two-hundred and twenty-five degrees (225°).

FIG. 6 depicts yet another embodiment, wherein a cap 470 has a cavity 475 defining an internal surface 478 configured to produce a plurality of obtuse angles θ2, β2. Therein, a first obtuse angle θ2 is produced by and between a sloping surface 486 and an underside surface 488 of the cavity 475. A second obtuse angle β2 is produced by and between the sloping surface 486 and a cylindrical bore 484 formed along the internal face of the cavity 475. In the described embodiment, the first obtuse angle θ2 is greater than about one-hundred and thirty-five degrees (135°) while the second obtuse angle β2 is also greater than about one-hundred and thirty-five degrees (135°).

In summary, the teachings of the present disclosure provide a new and useful sealing interface between a dead-end or other fitting and a sealing cap which facilitates high-velocity fluid flow to adequately clean the internal surfaces of residue and harmful bacteria. The disclosure describes a novel cap geometry which provides a rim portion having a mating surface configured to receive a compliant gasket along the mating surface and a central cover portion integrally formed with the rim portion. The cavity defines a cylindrical bore and an arcuate internal surface enclosing the cylindrical bore wherein the arcuate internal surface defines a threshold radius configured to prevent stagnation of a fluid induced to flow through the fitting and along the arcuate internal surface during cleaning operations.

Additional embodiments include any one of the embodiments described above, where one or more of its components, functionalities or structures is interchanged with, replaced by or augmented with one or more of the components, functionalities or structures of a different embodiment described above.

It should be understood that various changes and modifications to the embodiments described herein will be apparent to those skilled in the art. Such changes and modifications can be made without departing from the spirit and scope of the present disclosure and without diminishing its intended advantages. It is therefore intended that such changes and modifications be covered by the appended claims.

Although several embodiments of the disclosure have been disclosed in the foregoing specification, it is understood by those skilled in the art that many modifications and other embodiments of the disclosure will come to mind to which the disclosure pertains, having the benefit of the teaching presented in the foregoing description and associated drawings. It is thus understood that the disclosure is not limited to the specific embodiments disclosed herein above, and that many modifications and other embodiments are intended to be included within the scope of the appended claims. Moreover, although specific terms are employed herein, as well as in the claims which follow, they are used only in a generic and descriptive sense, and not for the purposes of limiting the present disclosure, nor the claims which follow.

What is claimed is:

1. A hygienic cap for sealing a fitting, comprising:
a rim portion defining a mating surface configured to receive a compliant gasket along the mating surface; and
a central cover portion integrally formed with the rim portion, the cover portion defining a cavity having a threshold radius defined by an arcuate internal surface of the cavity,
wherein the arcuate internal surface encloses one end of the cavity,
wherein the threshold radius is configured to inhibit stagnation of a cleaning fluid along the arcuate internal surface during cleaning operations.

2. The hygienic cap of claim 1, wherein the cavity further defines a threshold length configured to enable the cleaning fluid to flow across the compliant gasket while minimizing head loss in the cleaning fluid, and wherein the threshold radius is configured to maintain a substantially constant velocity of the cleaning fluid along the arcuate internal surface during the cleaning operations.

3. The hygienic cap of claim 2, wherein the cavity further defines a cap diameter that corresponds to a gasket diameter.

4. The hygienic cap of claim 1, wherein the threshold radius is greater than about at least one-quarter inches (¼ or 0.25 inches).

5. The hygienic cap of claim 1, wherein the threshold radius is greater than about at least one sixteenth inches ( 1/16 or 0.0625 inches).

6. The hygienic cap of claim 2, wherein the threshold length is greater than about at least one-sixteenth inches ( 1/16 or 0.0625 inches).

7. The hygienic cap of claim 2, wherein the threshold length is greater than about at least one-thirty seconds of an inch ( 1/32 or 0.03 inches).

8. A hygienic cap for sealing a fitting, comprising:
a rim portion defining a mating surface configured to receive a compliant gasket along the mating surface; and,
a central cover portion integrally formed with the rim portion, the cover portion forming a cavity defining at least one obtuse angle along an internal surface of the cavity,
wherein the arcuate internal surface encloses one end of the cavity,
wherein the at least one obtuse angle is configured to inhibit stagnation of a cleaning fluid along the arcuate internal surface during cleaning operations.

9. The hygienic cap of claim 8, wherein the cavity further defines a threshold length configured to enable the cleaning fluid to flow across the compliant gasket while minimizing head loss in the cleaning fluid.

10. The hygienic cap of claim 9, wherein the cavity further defines a cap diameter that corresponds to a gasket diameter.

11. The hygienic cap of claim 8, further comprising a second obtuse angle adjacent the first obtuse angle, the second obtuse angle formed by and between a first sloping surface along the underside of the central cover portion and a cylindrical bore along an internal face of the cavity.

12. The hygienic cap of claim 11, wherein the second obtuse angle is greater than the first obtuse angle.

13. The hygienic cap of claim 12, wherein the first obtuse angle is greater than about one-hundred and thirty-five degrees (135°), and wherein the second obtuse angle is greater than about two-hundred and twenty-five degrees (225°).

14. The hygienic cap of claim 9, wherein the threshold length is greater than about at least one-sixteenth inches ( 1/16 or 0.0625 inches).

15. The hygienic cap of claim 14, wherein the threshold length is greater than about at least one-thirty seconds of an inches ( 1/32 or 0.03 inches).

16. A method of manufacturing a hygienic cap for sealing a fitting, the method comprising:
structuring a rim portion to define a mating surface to receive a compliant gasket along the mating surface; and
structuring a central cover portion to be integrally formed with the rim portion;
structuring the central cover portion to define a cavity comprising a threshold radius defined by an arcuate internal surface of the cavity,
structuring the arcuate internal surface to enclose one end of the cavity,
wherein the threshold radius is configured to inhibit stagnation of a cleaning fluid along the arcuate internal surface during cleaning operations.

17. The method of claim 16, further comprising structuring the cavity to define a threshold length configured to enable the cleaning fluid to flow across the compliant gasket while minimizing head loss in the cleaning fluid.

18. The method of claim 17, further comprising structuring the cavity to define a cap diameter that corresponds to a gasket diameter.

19. The method of claim 16, further comprising structuring the threshold radius to be greater than about at least one-quarter inches (¼ or 0.25 inches).

20. The method of claim 16, further comprising structuring the threshold radius to be greater than about at least one sixteenth inches ( 1/16 or 0.0625 inches).
